(19) United States
(12) Patent Application Publication
(10) Pub. No.: US 2008/0132584 A1
(43) Pub. Date: Jun. 5, 2008

(54) Biofunctional Nanoprobes

(75) Inventors: David E. Luzzi, Wallingford, PA (US); Evan Goulet, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees of the University of Pennsylvania, Philadelphia, PA (US)

(21) Appl. No.: 11/720,562

(22) PCT Filed: Dec. 2, 2005
(86) PCT No.: PCT/US05/43933

§ 371 Date: Oct. 18, 2007

Related U.S. Application Data
(60) Provisional application No. 60/632,509, filed on Dec. 2, 2004.

(51) Int. Cl. A61K 47/00; B32B 3/10; C12N 9/00; C12N 5/00

(52) U.S. Cl. 514/770; 435/183; 428/357; 435/325; 977/747

(57) ABSTRACT

Biofunctional nanoprobes are disclosed having nanoscale dimensions enabling the non-destructive penetration of lipid membranes. They are functionalized to perform a bio-chemical process using bio-compatible, porous coating in which enzymes are structurally constrained.

### FIELD OF THE INVENTION

The invention relates to biofunctional nanoprobes comprising nanotubes coated with biocompatible coatings capable of transporting and delivering bioreactive or other bioactive molecules. Methods of making the nanoprobes and methods of delivery and use are also disclosed.

### BACKGROUND OF THE INVENTION

There is a need for more effective research solutions for programs researching disease or injury processes or chemical interaction processes in which the ability to modify the interior of a cell without damaging the cell membrane is important. The present invention is directed, inter alia, to this important goal.

### SUMMARY OF THE INVENTION

The present invention is directed to hybrid materials, which can function as nanoprobes, comprising a nanotube at least partially coated with a biofunctional coating. Preferably, the coating is capable of absorbing bio-reactive molecules, especially via steric interaction. In one embodiment, the coating may comprise colloidal silica. The nanotubes may be multi-walled or double-walled nanotubes. The nanotubes may comprise C₆₀ molecules within its interior.

The nanoprobes may have coatings that are porous. The one embodiment, the coating may be colloidal silica that preferably comprises generally spherical silica particles. A medicament or marking enzyme may also comprise a part of the coating. A medicament or marker may also be contained within the interior of the nanotube.

The invention also discloses methods comprising partially coating a nanotube with a bio-functional coating and contacting a lipid membrane with said coated nanotube. Another method that may be preferred comprises partially coating a nanotube with colloidal silica and penetrating a lipid membrane with said coated nanotube. It will be appreciated that there are methods comprising partially coating a nanotube with colloidal silica, imbibing said silica with a bio-reactive molecule, contacting a lipid membrane with the coated nanotube, and delivering said molecule to said lipid membrane. Some method embodiments comprise partially coating a nanotube with colloidal silica, imbibing said silica with a bio-reactive molecule, passing through a lipid membrane with said coated nanotube, and delivering said molecule.

### BRIEF DESCRIPTION OF THE DRAWINGS

FIG. 1 displays the absorption monitoring of horseradish peroxidase (HRP) reaction for one embodiment.

FIG. 2 displays the absorption monitoring of HRP reaction, for one embodiment.

FIG. 3 displays the reactivity corrected for MWNTs settling.

FIG. 4 depicts an embodiment of the invention with a tip having Ludox®/HRP coating.

FIG. 5 depicts another embodiment of the invention with a tip having Ludox®/HRP coating.

FIG. 6 depicts another embodiment of the invention with a tip having Ludox®/HRP coating.

FIG. 7 depicts an embodiment of the invention seen under fluorescence microscopy.

### DETAILED DESCRIPTION OF PREFERRED EMBODIMENTS

Embodiments of the present invention provide nanoprobes that introduce small quantities of a substance into a cell or the cell nucleus and either leave this substance behind or remove the substance after controllable intervals. It is foreseeable that substance delivered may be pharmaceutically beneficial such as a medicament. Other materials, such as markers, reactive moieties or other things having biological activity or which may be useful in research or therapeutics may also be employed. This delivery is characterized by minimal disruption of the lipid membrane of the cell or cell nucleus. Embodiments of the present invention allow for the delivery of many specified substances into a cell or a section of a cell without killing the cell or damaging the cell to an experimentally or chemically-significant amount. It also may be preferred to use certain embodiments of the invention as single cell nanoprobes for biomedical research.

To these ends, the present invention provides hybrid materials which can be utilized as biofunctional, nanoscopic probes, or nanoprobes. These comprise a nanotube at least partially coated with a biofunctional coating capable of absorbing bio-reactive molecules. Embodiments of the invention may also be described as comprising a nanotube having a coating capable of imbibing or absorbing bio-reactive molecules, said coating comprising colloidal silica. The nanotube component may be described as tubular or solid, high-aspect-ratio fiber with diameter between 1 and 100 nm. The nanotubes suitable for the present invention may be single walled (SWNT), double walled (DWNT), multi-walled (MWNT), or nanotubes modified using techniques known in the art. An example of a modified nanotube is one that comprises Buckminster fullerene, or C₆₀ balls without its interior.

In one embodiment, methods have been developed to generate biofunctional coatings at least partially covering nanotubes. The coating may be porous or meso-porous. The porous coating may preferably comprise silica or spherical silica particles. The porous nature of the coating lends itself to steric entrapment of bio-reactive molecules. Also envisioned are coating comprising marking enzymes such as horseradish peroxidase. These molecules may then be introduced into a lipid membrane, cell, or vesicle by using the nanoprobe as an invasive, but non-disruptive probe. It is understood that there may be coatings capable of absorbing molecules found in a lipid membrane or cell and extracting the molecule using the nanoprobe.

A suitable bio-reactive molecule may be horseradish peroxidase (HRP). HRP reduces peroxide, creating a radical oxygen. It then catalyzes the oxidation of 2,2′-Azino-bis(3-ethylbenzothiazoline-6-sulfonic acid) (ABTS) among other molecules. This oxidation of ABTS produces an absorption in solution at 735 nm, which is easily monitored via absorption spectroscopy.

There are also provided methods comprising partially coating a nanotube with colloidal silica, imbibing said silica with a bio-reactive molecule, contacting the coated nanotube with a lipid membrane, and delivering said molecule to said lipid membrane. The delivery method may also comprise partially coating a nanotube with colloidal silica, imbibing said silica with a bio-reactive molecule, passing through a lipid membrane with said coated nanotube, and delivering said molecule. As discussed previously, there are methods of partially coating a nanotube with a biofunctional coating; passing the coated nanotube through a lipid membrane, cell, or vesicle; and extracting a molecule from the interior of the lipid membrane, cell, or vesicle.

One skilled in the art may use some embodiments by utilizing atomic force microscope (AFM) technology for force sensing and fine position of the nanoprobe, as well as the longitudinal penetration translations. Light microscopes and micron-resolved mechanical control may be also be used so may other control and sensing modalities.

The embodiments of the present invention may be used to transport into a lipid membrane, cell, or vesicle a substance from the interior of a tubular fiber or nanotube. The delivery may be one-way. The development of fluidics at the sub-micron scale may be required in facilitating this transport.

Some embodiments may use the nanoprobes to deliver a substance that is a component of the coating on the exterior side-walls and/or the tip of the probe. Two technologies may be used for the production of bio-functional materials in which active enzymes are sterically-confined, yet active; one is a polymer-based composite, the other a sol-gel ceramic composite. The remaining technology development may be the conversion of these bulk materials into coatings on the fibers, which could involve chemical reaction development.

This invention also provides embodiments where the substance to be delivered is covalently bonded to the exterior of the fiber through a chemically functional ligand. This may involve the direct functionalization of the fiber surface with bio-active molecules via chemical ligands. Other potentially useful configurations of the system provide provisions for creating an electrostatic potential between the probe and the cell interior and/or the encapsulation of optically-emitting molecules (especially in the near-IR) within the lumen of tubular fibers as a means for probe location and optical stimulation of the cell.

### EXAMPLES

MWNTs are refluxed for three hours in concentrated nitric acid at 85° C.-100° C. under constant stirring. This mixture is then centrifuged and washed until the pH of the resulting suspension measure approximately 6.0. At this point the suspension is sonicated in a bath sonicator for approximately 15 minutes to reduce aggregation.

The MWNTs are coated with polyethyleneimine (PEI). A solution of 5 mM PEI in de-ionized water is made. To this the acid-treated MWNTs are added. This suspension is sonicated for 24 hours in a bath sonicator. The suspension is then centrifuged and washed twice to remove excess PEI. The MWNTs are suspended in phosphate buffer solution (PBS) at a pH of 7.2.

To this suspension of PEI-coated MWNTs is added HRP at a concentration of 1 mg/mL. Ludox® Colloidal Silica (provided by Grace Davison) SM-30 colloidal silica is also added at a silica weight concentration of less than 1%. This mixture is placed in a refrigerator at 4° C. under constant stirring for 5 days. At the end of 5 days, the mixture is centrifuged and washed twice at 4° C. with PBS. This step is intended to remove as much excess colloidal silica as possible.

The suspension is then filtered with copious amounts of PBS. After each filtration step, the filtrate is evaluated on the absorption spectrometer for HRP reactivity. The standard method that has been developed is as follows. In a 10 mm quartz cuvette, 3 mL of 0.1 M ABTS in PBS is mixed with 5 μL of 0.1% H₂O₂. The instrument is set-up to monitor the absorption of the solution at 735 nm. The ABTS/ H₂O₂ solution is used to zero the instrument at 735 nm. Then 1 mL of filtrate is added to the cuvette and the reaction is monitored. When the reactivity of the filtrate is deemed negligible, the suspension containing the bio-functional MWNTs can be tested with the confidence that free HRP in the PBS is not contributing significantly to the reaction. The absorption spectroscopy of the bio-functional MWNTs is carried out similar to the evaluation described above.

TEM images are also obtained of the bio-functional MWNTs. The TEM samples are prepared on holey carbon, copper grids. The suspension containing the MWNTs is diluted at a ratio of 1:10 and sonicated for approximately 5 seconds. Then 10 μL of this dilute suspension is applied to the TEM grid. The drop of suspension is allowed to sit for approximately 15 minutes before being wicked away with a small piece of glass fiber filter paper. The TEM grid is then stored in a vacuum desiccator until TEM inspection. All TEM inspections are carried out at either 80 kV or 100 kV.

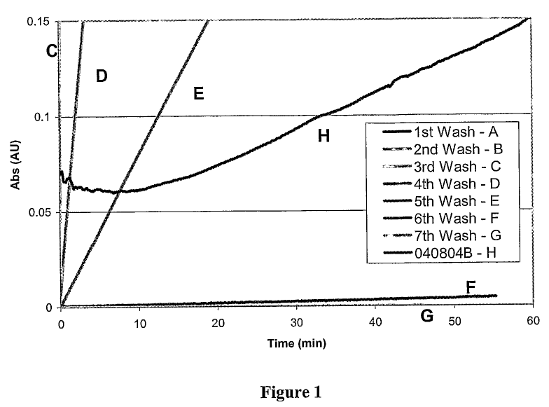

Absorption Spectroscopy Results: Two batches of bio-functional MWNTs were produced by the method above. The results for one batch are shown in FIG. 1. The seven washes are shown in the figure to illustrate the washing process. Each wash showed successively less actively than the previous wash. The H curve represents the activity of the bio-functional MWNTs of one batch in a suspension of PBS. One can see that the activity is clearly greater than that of either the 6th or 7th washes. This is indicates that the greatest source for HRP activity are the bio-functional MWNTs.

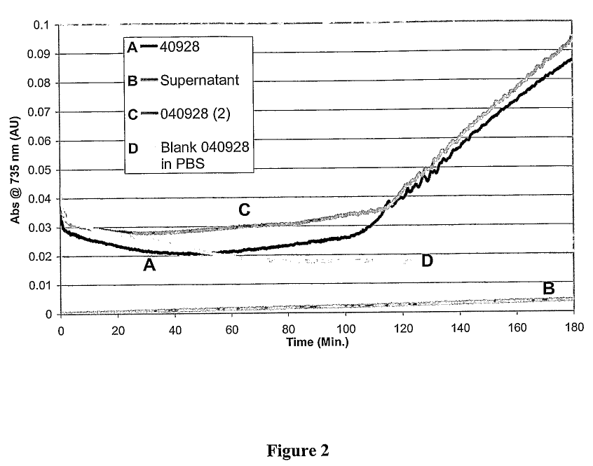

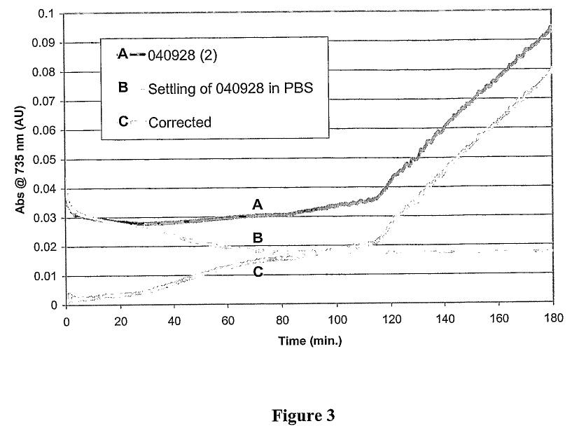

Similar results were obtained with another batch. The absorption spectroscopy results for this batch are shown in FIG. 2. The B curve represents the reactivity of the supernatant obtained after the final wash. The C and A curves represent two different data sets from this batch, while the D curve shows the settling of the bio-functional MWNTs in PBS without ABTS or H₂O₂ present. The settling of the MWNTs was then subtracted from the A curve as shown in FIG. 3. The reaction for this batch was monitored for 180 minutes, and it showed two regions of activity. The first region has a shallow slope that occurs from the start of the reaction, and the second region shows a much steeper slope that occurs after approximately 110 minutes. The reason for the two different stages of reactivity is unexplained as of yet.

Both of these batches showed significantly greater activity than the final wash filtrate. This indicates that the HRP is immobilized on the MWNTs by the coating of Ludox® silica particles. However, there is present a significant amount of silica agglomerates that may be lending to the overall activity of the batch. Currently there is no method for removing the excess silica agglomerates.

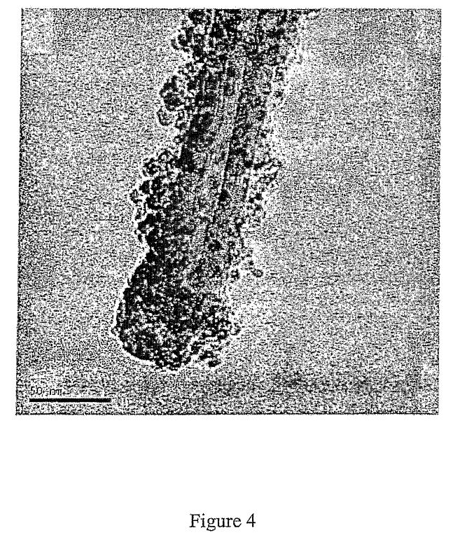

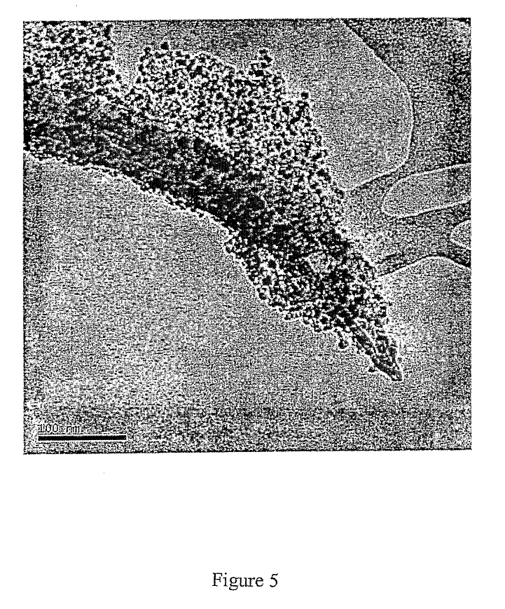

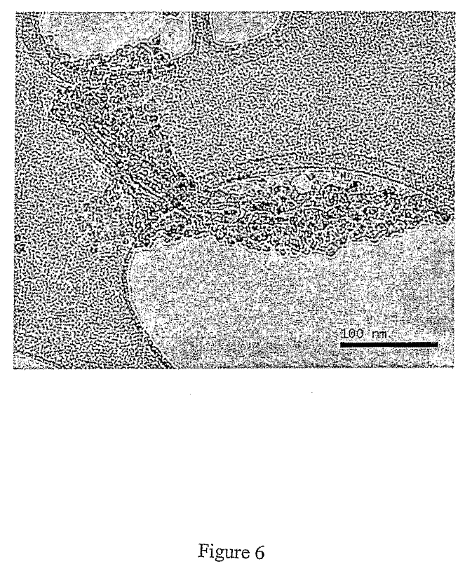

TEM Results: TEM evaluation was performed on both of the batches mentioned above. Both batches exhibited MWNTs coated with Ludox(® particles. Most of the MWNTs were isolated, however some tangles of MWNTs were observed. A majority of the bio-functional MWNTs were less than 1 μm in length. This fact is probably due to the long period of sonication that is required during the PEI coating step. The HRP that is entrapped in the Ludox® coating is not visible within the TEM. This is probably due to the fact that the enzyme does not have sufficient density to cause contrast between the MWNTs and silica particles. FIGS. 4 and 5 show typical Ludox®-coated MWNTs from the first batch. FIG. 6 shows a typical MWNT from the second batch.

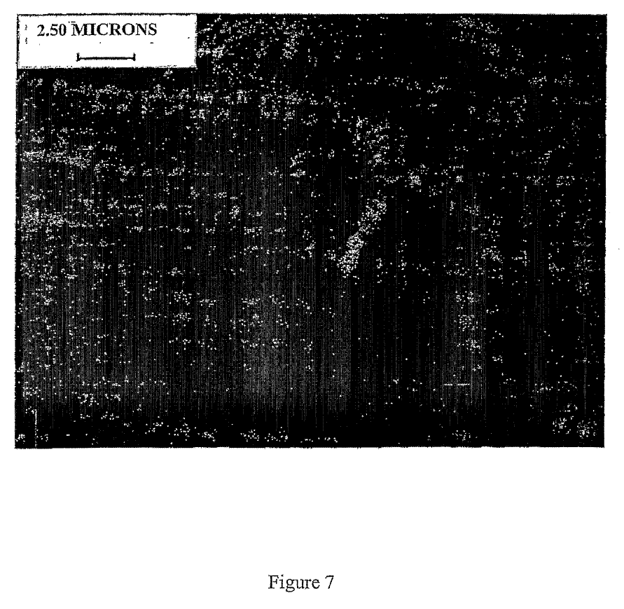

Fluorescence microscopy was used to confirm that the enzyme is bound to the nanoprobe as shown in FIG. 7. BRP was functionalized with a fluorescent tag prior to creation of the nanoprobe. After nanoprobe synthesis, the nanoprobe was repeatedly washed to remove all unbound BRP. The nanoprobe was then imaged in a fluorescence microscope; the fluorescent image is shown in the figure. The strong localization of the fluorescence signal to the high-aspect ratio object in the image is consistent with the functional HRP being bound within the colloidal silica coating on the nanotube.

What is claimed is:

1. A hybrid material comprising a nanotube at least partially coated with a biofunctional coating capable of absorbing bio-reactive molecules.

2. A hybrid material comprising a nanotube having a coating comprising colloidal silica capable of imbibing bio-reactive molecules.

3. The hybrid material of claim 2 wherein said nanotube is a multi-walled nanotube.

4. The hybrid material of claim 2 wherein said nanotube is a double-walled nanotube.

5. The hybrid material of claim 2 wherein said nanotube comprises C₆₀ molecules within its sidewalls.

6. The hybrid material of claim 2 wherein said coating comprising an enzyme.

7. The hybrid material of claim 2 wherein said coating comprises horseradish peroxidase.

8. The hybrid material of claim 2 wherein said coating is porous.

9. The hybrid material of claim 2 wherein said colloidal silica is spherical silica particles.

10. The hybrid material of claim 2 wherein said coating further comprises a medicament.

11. A method comprising:
partially coating a nanotube with a bio-functional coating and
contacting a lipid membrane with said coated nanotube.

12. A method comprising:
partially coating a nanotube with colloidal silica and
penetrating a lipid membrane with said coated nanotube.

13. A method of delivery comprising:
partially coating a nanotube with colloidal silica;
imbibing said silica with a bio-reactive molecule;
contacting the coated nanotube with a lipid membrane; and
delivering said molecule to said lipid membrane.

14. A method of delivery comprising:
partially coating a nanotube with colloidal silica;
imbibing said silica with a bio-reactive molecule;
passing through a lipid membrane with said coated nanotube; and
delivering said molecule.
